(19) United States
(12) Patent Application Publication
(10) Pub. No.: US 2021/0177921 A1
(43) Pub. Date: Jun. 17, 2021

(54) TARGETING MOIETY-DECORATED ONCOLYTIC VIRUSES

(71) Applicant: VIROGIN BIOTECH CANADA LTD, Vancouver (CA)

(72) Inventors: William Jia, Burnaby (CA); Dmitry V. Chouljenko, Vancouver (CA); Xiaohu Liu, Vancouver (CA); Yanal M. Murad, Vancouver (CA); Xuexian Bu, Vancouver (CA)

(21) Appl. No.: 16/764,746

(22) PCT Filed: Nov. 16, 2018
(86) PCT No.: PCT/US2018/061687

§ 371 Date: May 15, 2020

Related U.S. Application Data
(60) Provisional application No. 62/587,103, filed on Nov. 16, 2017.

(51) Int. Cl. A61K 35/763; C12N 7/00; C07K 16/08; C07K 16/30; A61P 35/00

(52) U.S. Cl. CPC A61K 35/763; C12N 7/00; C07K 16/087; C07K 16/30; C07K 16/3007; A61K 2039/505; C12N 2710/16033; C12N 2710/24133; C12N 2710/10033; C07K 2317/31; C07K 2317/569; A61P 35/00

(57) ABSTRACT

Compositions and methods are provided for treating cancer comprising a composition comprising an oncolytic virus and a targeting moiety on the surface of the virus, wherein the oncolytic virus does not encode or express the targeting moiety within various aspects such compositions and methods can be used to treat a wide variety of cancers (e.g., gastrointestinal cancer, such as esophageal, stomach and colon cancers).

### CROSS-REFERENCE TO RELATED APPLICATION

This patent application claims the benefit under 35 U.S.C. § 119(e) of U.S. Provisional Patent Application No. 62/587,103 filed Nov. 16, 2017, which application is incorporated herein by reference in its entirety for all purposes.

### FIELD OF THE INVENTION

The present invention relates generally to oncolytic viruses that have targeting moieties on their surface.

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “VIR0405_ST25.txt”, a creation date of Nov. 16, 2017, and a size of 4.30 KB. The Sequence Listing filed via EFS-Web is part of the specification and is incorporated in its entirety by reference herein.

### BACKGROUND

Oncolytic virotherapy has been recognized as a promising new therapeutic approach for cancer treatment because oncolytic viruses cause strong tumor oncolysis and induce a systemic tumor-specific immunity while causing significantly fewer side effects than chemotherapy or radiation treatments.

Among the various OVs, herpes simplex virus type 1 (“HSV-1”) based OVs are the farthest advanced, e.g., a herpes virus-based OV (T-Vec) has been approved by the U.S. FDA for the treatment of melanoma. Representative examples of HSV vectors include those described in U.S. Pat. Nos. 7,223,593, 7,537,924, 7,063,835, 7,063,851, 7,118,755, 8,277,818, and 8,680,068.

One difficulty of treating cancers however, is the ability to directly target and kill the cancer, as opposed to other non-cancerous tissues and cells. One method that has been developed to meet this need is attenuation of the oncolytic virus, e.g., by deleting or altering certain viral genes or gene regions which make the virus safer and more cancer-specific.

Other methods that have been developed for making an oncolytic virus more cancer specific include, for example: 1) transductional targeting (e.g., modifying the viral coat protein to specifically target cancer cells, while reducing the likelihood of entry into non-cancerous cells); and 2) non-transductional targeting (e.g., modifying the viral genome to replicate only in cancer cells, or transcriptionally controlling critical parts of the viral genome to only replicate in cancers, e.g., under the control of a tumor-specific promoter).

One difficulty however with targeting specific populations of cancer cells, is that cancers which present themselves in a clinical setting rarely exist as a single monoclonal population (see, e.g., Shen, Michael M. “The complex seeds of metastasis: Analyses of prostate-cancer metastases reveal a complex cellular architecture, and show that secondary sites can be seeded by multiple cell populations derived from both the primary tumor and other metastases.” Nature, vol. 520, no. 7547, 2015). Hence, while it may be possible to target a monoclonal population of tumor cells, in a clinical setting such treatments can meet with only limited success.

The present invention overcomes shortcomings of current commercial oncolytic viruses, and further provides additional unexpected benefits.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

Briefly stated, the invention provides compositions and methods for treating cancer with an oncolytic virus, utilizing a novel strategy, namely, to provide specific targeting of the OV for only the first cycle of viral replication, and thereafter, allowing subsequent generations of the OV to replicate in cells according to how it is (or has been) genetically constructed. For example, a first generation of an OV can be constructed to be decorated on its surface with a targeting moiety (as described in more detail below). Subsequent generations of the OV which do not express the targeting moiety will target and replicate in cells according to the genetic nature of the OV

The techniques described herein can be employed with a number of different oncolytic viruses, including for example, adenovirus, herpes simplex virus (HSV), influenza virus, rhabdovirus (e.g. vesicular stomatitis virus (VSV)) and pox viruses such as vaccinia virus.

Within one embodiment of the invention the targeting moiety is a tumor antigen specific polypeptide or antibody bound to an envelope protein. Within certain embodiments, the envelope protein is not responsible for OV infection (e.g., in the case of HSV, gC or gG). Within alternative embodiments, the envelope protein is (at least in part) responsible for OV infection (e.g., in the case of HSV, gD). For example, the envelope protein can be bound (through the techniques described herein) to tumor specific antibodies in vitro to generate an antibody decorated oHSV for tumor targeting. A key advantage of this strategy lies in its versatility because the oHSV can be combined with any tumor specific antibodies for each different tumor based on the cell surface proteins highly expressed for individual patients, which allows for more precise targeting and a more personalized approach for each patient. Furthermore, because the antibody is not genetically encoded within the viral genome, the progeny virus from the initial infected tumor cells are not restricted only to tumor cells bearing the cell surface marker and can infect all cells within the tumor mass, which should greatly enhance tumor destruction.

An alternative strategy is to use a bispecific antibody to target both an OV envelope protein and a tumor surface antigen. Mixing the bispecific antibody with OV coats the virus surface with the antibody and primes the virus to preferentially target tumor cells. After replication in the tumor mass, the progeny virus also lose the antibody and are not restricted to a particular tumor cell target, thus allowing for a much broader range of infectivity to kill a variety of tumor-associated cells.

Within particularly preferred embodiments of the invention, tumor targeting could be further enhanced by engineering the bispecific antibody to bind, e.g., HSV glycoprotein D, thus detargeting the virus from its natural receptors by altering gD-mediated tissue tropism. A similar approach using anti-gD antibody could be utilized to render the virus more cancer-specific after the virus has already been modified to display a tumor-specific antibody.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 diagrammatically illustrates the comparison of a representative single domain antibody, heavy chain antibody, and a traditional antibody.

FIG. 2 illustrates a representative sandwich ELISA assay that can be used to quantify the amount of the anti-gC-anti-CEACAM6 bispecific antibody bound to oHSV. Quantification of the efficiency of bispecific antibody conjugation to oHSV can be conducted by applying the virus lysate to ELISA plates coated with anti-llama or anti-gGantibodies followed by incubation with detection probes. As should be readily evident given the disclosure provided herein, similar ELISA assays may be similarly run to quantify other antibodies (e.g., bispecific antibodies wherein one aspect of the antibody binds to an envelope protein selected from the group consisting of gB, gC, gE, gI, gJ, gK, gM, gN, UL20, UL24, UL43, UL45, UL56, and US9. Within certain preferred embodiments, one aspect of the bispecific antibody binds to gD.

FIG. 3 illustrates a representative ELISA assay that can be used to quantify the amount of the SpyTag-CEACAM6 antibody bound to SpyCatcher-contained oHSV. Quantification of the efficiency of SpyCatcher/SpyTag conjugation to oHSV can be conducted by adding a detection antibody or probe to ELISA plates coated with the virus lysate of SpyCatcher/SpyTag-anti-CEACAM6-decorated oHSV.

FIG. 4 provides a representative illustration of conjugation between recombinant SpyCatcher and SpyTag anti-CEACAM6.

FIG. 5 provides a representative illustration which quantifies the conjugation between SpyCatcher-expressing virus and SpyTag anti-CEACAM6 by ELISA.

FIG. 6 provides a representative illustration of virus retargeting in vitro using SpyCatcher/SpyTag.

FIG. 7 provides a representative illustration of an anti-gD-anti-CEACAM6 bispecific antibody and an assay of the interaction between oHSV-1 with the bispecific antibody.

FIG. 8A provides a diagrammatic illustration of SpyCatcher fused in frame into gG and gC. FIG. 8B provides an illustrations of cell lysates on SDS-PAGE.

### DETAILED DESCRIPTION OF THE INVENTION

The present invention may be understood more readily by reference to the following detailed description of preferred embodiments of the invention and the Examples included herein.

The term “targeting moiety” as used herein refers to a molecule, complex or aggregate, that binds specifically or selectively to a target molecule, cell, particle, tissue or aggregate. In preferred embodiments, a targeting moiety is an antibody as described in more detail below. Other representative examples of targeting moieties include aptamers, avimers, receptor-binding ligands, and nucleic acids. The terms “targeting moiety” and “binding moiety” are used synonymously herein.

The term “antibody” or “antibodies” refers to both full-length immunoglobulins (i.e., naturally occurring or recombinantly formed whole molecules) (e.g., an IgG antibody such as IgG1, IgG2a, IgG3, IgG4 (and IgG4 subforms), IgA isotypes, IgE and IgM) or an immunologically active (i.e., specifically binding) portion of an immunoglobulin molecule, such as an antibody fragment or segment. Representative antibody fragments or segments include separate heavy chains, light chains, and portions of an antibody such as F(ab′)₂, F(ab)₂, Fab′, Fab, Fv, scFv (single chain Fv) and the like, including the half-molecules of IgG4 (see van der Neut Kolfschoten et al. (Science 2007; 317 (14 September): 1554-1557). Antibody fragments or segments also include immunologically active minimal recognition units consisting of the amino acid residues that mimic the hypervariable region, such as CDRs. Antibody fragments or segments can be produced by enzymatic or chemical separation of intact immunoglobulins, or, by recombinant techniques. The term “antibody” should also be understood to include one or more immunoglobulin chains that are chemically conjugated to, or expressed as fusion proteins along with other proteins.

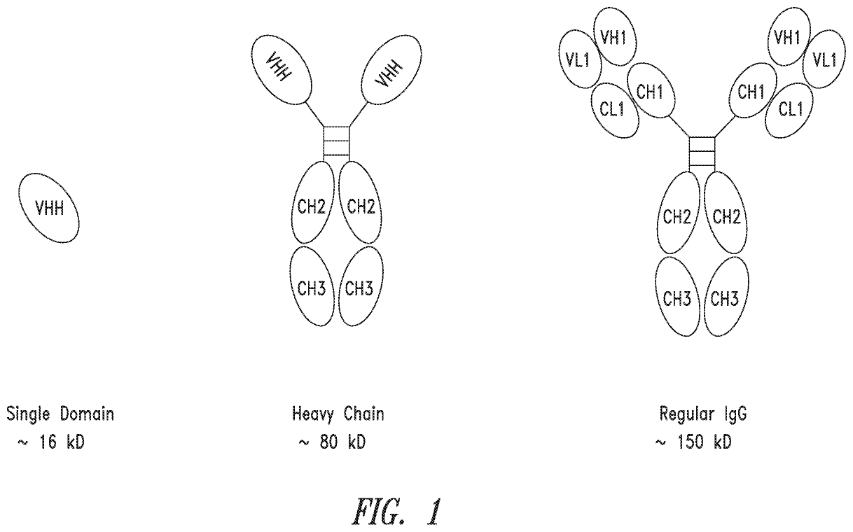

The term “antibody” also includes single domain antibodies (sdAbs) or nanobodies, and bispecific or bifunctional antibodies (e.g., an artificial hybrid antibody having two different heavy/light chain pairs and two different binding sites). SdAbs are comprised of a single monomeric variable region and may be derived from either heavy chains or light chains. One advantage of sdAbs over traditional monoclonal antibodies or other antibodies such as scFv or diabodies is the significantly smaller size (^{˜}2 nm) of single domain antibodies (FIG. 1), thus making them less likely to interfere with the functions of essential glycoproteins on the viral envelope due to steric hindrance. In addition, sdAbs and nanobodies exhibit high affinity towards their targets and excellent biophysical properties such as thermal stability.

FIG. 1 is provided to illustratively compare a representative single domain antibody, heavy chain antibody, and a traditional antibody.

Covalent Binding Pair (“CBP”, or individually, “CPB1” and “CPB2”) refers to two molecules which have high specificity for binding each other. Within preferred embodiments of the invention the CPB should: 1) have high specificity for each other; and 2) a very low specificity for molecules which occur or can be found naturally in a human subject. Within particularly preferred embodiments of the invention the CPB bind to each other covalently. Representative examples of CPB include the SpyTag/SpyCatcher pair (see, e.g., Reddington and Howarth, “Secrets of a covalent interaction for biomaterials and biotechnology: SpyTag and SpyCatcher,” Current Opinion in Chemical Biology, 2015:29:94-99; see also, U.S. Pat. No. 9,547,003 entitled “Peptide tag systems that spontaneously form an irreversible link to protein partners via isopeptide bonds”; both of which are incorporated by reference in their entirety). Other representative examples include those disclosed by: Zakeri and Howarth, “Spontaneous Intermolecular Amide Bond Formation between Side Chains for Irreversible Peptide Targeting”, 4526 J. Am. Chem. Soc. 2010, 132, 4526-4527; and by Tan L L., Hoon S S, Wong F T (2016) “Kinetic Controlled Tag-Catcher Interactions for Directed Covalent Protein Assembly. PloS ONE 11 (10):e0165074doi:10.137/journal.pone.0165074, both of which are incorporated by reference in their entirety. The term “oncolytic virus” refers generally to any virus capable of replicating in and killing tumor cells. Within certain embodiments the virus can be engineered in order to more selectively target tumor cells. Representative examples of oncolytic viruses include without limitation, adenovirus, coxsackievirus, H-1 parvovirus, herpes simplex virus (HSV), influenza virus, measles virus, Myxoma virus, Newcastle disease virus, parvovirus picornavirus, reovirus, rhabdovirus (e.g. vesicular stomatitis virus (VSV)), paramyxovirus such as Newcastle disease virus, picornavirus such as poliovirus or Seneca valley virus, pox viruses such as vaccinia virus (e.g. Copenhagen, Ind. Western Reserve, and Wyeth strains), reovirus, or retrovirus such as murine leukemia virus. Further representative examples are described in: U.S. Pat. Nos. 8,147,822 and 9,045,729 (oncolytic rhabdovirus/VSV); U.S. Pat. No. 9,272,008 (oncolytic Measles virus); U.S. Pat. Nos. 7,223,593, 7,537,924, 7,063,835, 7,063,851, 7,118,755, 8,216,564, 8,277,818, and 8,680,068 (oncolytic herpes virus vectors); and U.S. Pat. No. 8,980,246 (oncolytic vaccinia virus), all of which are incorporated by reference in their entirety.

“Treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

Representative forms of cancer include carcinomas, leukemia's, lymphomas, myelomas and sarcomas. Further examples include, but are not limited to cancer of the bile duct cancer, brain (e.g., glioblastoma), breast, cervix, colorectal, CNS (e.g., acoustic neuroma, astrocytoma, craniopharyogioma, ependymoma, glioblastoma, hemangioblastoma, medulloblastoma, menangioma, neuroblastoma, oligodendroglioma, pinealoma and retinoblastoma), endometrial lining, hematopoietic cells (e.g., leukemia's and lymphomas), kidney, larynx, lung, liver, oral cavity, ovaries, pancreas, prostate, skin (e.g., melanoma and squamous cell carcinoma) and thyroid. Cancers can comprise solid tumors (e.g., sarcomas such as fibrosarcoma, myxosarcoma, liposarcoma, chondrosarcoma and osteogenic sarcoma), be diffuse (e.g., leukemia's), or some combination of these (e.g., a metastatic cancer having both solid tumors and disseminated or diffuse cancer cells). Cancers can also be resistant to conventional treatment (e.g. conventional chemotherapy and/or radiation therapy).

Benign tumors and other conditions of unwanted cell proliferation may also be treated.

“Tumor antigen” or “tumor antigens” as utilized herein refers to antigens that presented by MHC class I or class II molecules on the surface of tumor cells. Antigens which are found only on tumor cells are referred to as “Tumor Specific Antigens” or “TSAs”, while antigens that are presented by both tumor cells and normal cells are referred to as “Tumor Associated Antigens” or “TAAs”. Representative examples of tumor antigens include, but are not limited to AIM-2, AIM-3, ART1, ART4, BAGE, β1,6-N, β-catenin, B-cyclin, BMI1, BRAF, BRAP, C13orf24, C6orf153, C9orfl12, CA-125, CABYR, CASP-8, cathepsin B, Cav-1, CD74, CDK-1, CEAmidkin, COX-2, CRISP3, CSAG2, CTAG2, CYNL2, DHFR, E-cadherin, EGFRvIII, EphA2/Eck, ESO-1, EZH2, Fra-1/Fosl 1, FTHL17, GAGE1, Ganglioside/GD2, GLEA2, Glil, GnT-V, GOLGA, gp75, gplOO, HER-2, HSPH1, IL13Ralpha, IL13Ralpha2, ING4, Ki67, KIAA0376, Ku70/80, LDHC, LICAM, Livin, MAGE-A1, MAGE-2, MAGE-A3, MAGE-86, MAPPK1, MART-1, MICA, MRP-3, MUC-1, MUM-1, Nestin, NKTR, NLRP4, NSEP1, NY-ES-01, OLIG2, p53, PAP, PBK, PRAME, PROX1, PSA, PSCA, PSMA, ras, RBPSUH, RTN4, SART1, SART2, SART3, SOX10, SOX11, SOX2, SPANXA1, SSX2, SSX4, SSX5, Survivin, TNKS2, TPR, TRP-1, TRP-2, TSGA10, TSSK6, TULP2, Tyrosinase, U2AF1L, UPAR, WT-1, XAGE2, and ZNF165.

Within certain embodiments of the invention CEACAM6 and EpCAM are utilized as surface markers for tumor targeting. Briefly, CEACAM6 (carcinoembryonic antigen-related cell adhesion molecule) is a cell surface glycoprotein which functions as an intercellular adhesion molecule. EpCAM (epithelial cell adhesion molecule) is a transmembrane glycoprotein which mediates homotypic cell-cell adhesion. EpCAM is highly expressed in most epithelial-derived neoplasms and has been used as a diagnostic and prognostic marker for a variety of carcinomas. EpCAM plays a role in carcinogenesis by promoting cell proliferation and metastasis and by transcriptionally upregulating oncogenes c-myc and cyclin A/E.

### A. Oncolytic Virus Vector

As noted above, the present invention provides oncolytic viruses which may be decorated with a targeting moiety. Briefly, an oncolytic virus is a virus that will lyse cancer cells (oncolysis), preferably in a selective manner. Viruses that selectively replicate in dividing cells over non-dividing cells are often oncolytic. Oncolytic viruses suitable for use herein include Herpes Simplex Viruses 1 and 2.

Herpes Simplex Virus (HSV) 1 and 2 are members of the Herpesviridae family, which infects humans. The HSV genome contains two unique regions, which are designated unique long (U_{L}) and unique short (U_{S}) region. Each of these regions is flanked by a pair of inverted terminal repeat sequences. There are about 75 known open reading frames. The viral genome has been engineered to develop oncolytic viruses for use in e.g. cancer therapy. Tumor-selective replication of HSV may be conferred by mutation of the HSV ICP34.5 (also called γ34.5) gene. HSV contains two copies of ICP34.5. Mutants inactivating one or both copies of the ICP34.5 gene are known to lack neurovirulence, i.e. be avirulent/non-neurovirulent and be oncolytic. Tumor-selective replication can also be achieved without deleting ICP34.5, but by microRNA-based regulation of gene expression, or, by using tumor-specific promoters to drive expression of selected viral genes.

Suitable oncolytic HSV may be derived from either HSV-1 or HSV-2, including any laboratory strain or clinical isolate. In some embodiments, the oHSV may be derived from one of laboratory strains HSV-1 strain 17, HSV-1 strain F, or HSV-2 strain HG52. In other embodiments, it may be derived from non-laboratory strain JS-1. Other suitable HSV-1 viruses include HrrR3 (Goldstein and Weller, J. Virol. 62, 196-205, 1988), G2O7 (Mineta et al. Nature Medicine. 1(9):938-943, 1995; Kooby et al. The FASEB Journal, 13(11):1325-1334, 1999); G47Delta (Todo et al. Proceedings of the National Academy of Sciences. 2001; 98(11):6396-6401); HSV 1716 (Mace et al. Head & Neck, 2008; 30(8):1045-1051; Harrow et al. Gene Therapy. 2004; 11(22):1648-1658); HF10 (Nakao et al. Cancer Gene Therapy. 2011; 18(3):167-175); NV1020 (Fong et al. Molecular Therapy, 2009; 17(2):389-394); T-VEC (Andtbacka et al. Journal of Clinical Oncology, 2015: 33(25):2780-8); J100 (Gaston et al. PloS one, 2013; 8(11):e81768); M002 (Parker et al. Proceedings of the National Academy of Sciences, 2000; 97(5):2208-2213); NV1042 (Passer et al. Cancer Gene Therapy. 2013; 20(1):17-24); G2O7-IL2 (Carew et al. Molecular Therapy, 2001; 4(3):250-256); rQNestin34.5 (Kambara et al. Cancer Research, 2005; 65(7):2832-2839); G47Δ-mIL-18 (Fukuhara et al. Cancer Research, 2005; 65(23):10663-10668); and those vectors which are disclosed in PCT applications PCT/US2017/030308 entitled “HSV Vectors with Enhanced Replication in Cancer Cells”, and PCT/US2017/018539 entitled “Compositions and Methods of Using Stat1/3 Inhibitors with Oncolytic Herpes Virus”, all of the above of which are incorporated by reference in their entirety.

The oHSV vector may have modifications, mutations, or deletion of at least one γ34.5 gene. In some embodiments, both genes are deleted, mutated or modified. In other embodiments, one is deleted and the other is mutated or modified. Either native γ34.5 gene can be deleted. In one embodiment, the terminal repeat, which comprises γ34.5 gene and ICP4 gene, is deleted. Mutations, such as nucleotide alterations, insertions and deletions render the gene inexpressible or the product inactive. The γ34.5 gene may be modified with miRNA target sequences in its 3′ UTR. The target sequences bind miRNAs that are expressed at lower levels in tumor cells than in their normal counterparts. In some embodiments, the modified or mutated γ34.5 gene(s) are constructed in vitro and inserted into the oHSV vector as replacements for the viral gene(s). When the modified or mutated γ34.5 gene is a replacement of only one γ34.5 gene, the other γ34.5 is deleted. The γ34.5 gene may comprise additional changes, such as having an exogenous promoter. Within further embodiments, the γ34.5 gene can be translationally regulated, e.g., via the addition of an exogenous 5′ UTR such as the rat FGF-2 5′ UTR. This 5′ UTR forms secondary hairpin structures that can be unwound in the presence of sufficient eukaryotic initiation factor (eIF)4E/eIF4F complexes, leading to translation initiation of the mRNA. The eIF4E protein, part of the eIF4F complex, is known to be overexpressed in a variety of cancer types. Within yet other embodiments of the invention, neurovirulence may be prevented without modification of γ34.5 gene by employing mutations which prevent the virus from entering neurons in the first place, for example, by deleting amino acids 31-68 of glycoprotein K.

The oHSV may have additional mutations, which may include disabling mutations e.g., deletions, substitutions, insertions), which may affect the virulence of the virus or its ability to replicate. For example, mutations may be made in any one or more of ICP6, CPO, ICP4, ICP27, ICP47, ICP 24, ICP56. Preferably, a mutation in one of these genes (optionally in both copies of the gene where appropriate) leads to an inability (or reduction of the ability) of the HSV to express the corresponding functional polypeptide. In some embodiments, the promoter of a viral gene may be substituted with a promoter that is selectively active in target cells or inducible upon delivery of an inducer or inducible upon a cellular event or particular environment. In particular embodiments, a tumor-specific promoter drives expression of viral genes essential for replication of HSV. In certain embodiments the expression of ICP4 or ICP27 or both is controlled by an exogenous promoter, e.g., a tumor-specific promoter. Exemplary tumor-specific promoters include survivin or telomerase; other suitable tumor-specific promoters may be specific to a single tumor type and are known in the art. Other elements may be present. In some cases, an enhancer such as NF-kB/OCT4/SOX2 enhancer is present, for example in the regulatory regions of ICP4 or ICP27 or both. As well, the 5′UTR may be exogenous, such as a 5′UTR from growth factor genes such as FGF.

The oHSV may also have genes and nucleotide sequences that are non-HSV in origin. For example, a sequence that encodes a prodrug, a sequence that encodes a cytokine or other immune stimulating factor, a tumor-specific promoter, an inducible promoter, an enhancer, a sequence homologous to a host cell, among others may be in the oHSV genome. Exemplary sequences encode IL12, IL15, OX40L, PD-L1 blocker or a PD-1 blocker. For sequences that encode a product, they are operatively linked to a promoter sequence and other regulatory sequences (e.g., enhancer, polyadenylation signal sequence) necessary or desirable for expression.

The regulatory region of viral genes may be modified to comprise response elements that affect expression. Exemplary response elements include response elements for NF-κB, Oct-3/4-SOX2, enhancers, silencers, cAMP response elements, CAAT enhancer binding sequences, and insulators. Other response elements may also be included. A viral promoter may be replaced with a different promoter. The choice of the promoter will depend upon a number of factors, such as the proposed use of the HSV vector, treatment of the patient, disease state or condition, and ease of applying an inducer (for an inducible promoter). For treatment of cancer, generally when a promoter is replaced it will be with a cell-specific or tissue-specific or tumor-specific promoter. Tumor-specific, cell-specific and tissue-specific promoters are known in the art. Other gene elements may be modified as well. For example, the 5′ UTR of the viral gene may be replaced with an exogenous UTR.

### B. Targeting Moieties

As noted above, the present invention provides targeting moieties that can be utilized to decorate the surface of an OV, preferably, an oHSV.

Within one embodiment of the invention, HSV-1 mutants are generated with deletion of the entire ectodomain of gC, gD or gG, or alternatively, with deletions in only portions of the ectodomain (e.g., less than 5, 10, 20, 30, 40 or 50%, of the entire domain). As an example, in the case of gC only the heparin sulfate binding portion of ectodomain 1 may be deleted. Replacing all or a portion of the ectodomain (e.g., of gC or gG) with tumor targeting agents (peptides or antibodies) leads to a reduction in virus binding to non-tumor cells (which also express heparan sulfate) and enhances viral affinity for tumor cells. Within yet other embodiments, the entire sequence of the ectodomain may be left, but one aspect of a CBP (e.g., SpyCatcher) can be inserted prior to, within or after the envelope coding domain (e.g., after the signal peptide).

HSV mutants with deletions in the ectodomains of envelope proteins (e.g., gC, gD or gG are readily generated by homologous recombination technology. Specifically, viral mutagenesis is performed using a lambda Red-mediated recombineering system implemented on the HSV-1 genome cloned into a bacterial artificial chromosome (BAC).

Within preferred embodiments the SpyCatcher peptide is linked into an entire ectodomain of gC, gD or gG, or, into a truncated gC, gD or gG for virus decoration with the SpyCatcher/SpyTag system. The SpyCatcher/SpyTag tagging system is derived from the CnaB2 domain of the fibronectin binding protein FbaB found in Streptococcus pyogenes. When mixed together, the N-terminal protein fragment SpyCatcher and C-terminal peptide SpyTag, split from CnaB2, specifically associate and spontaneously form an isopeptide bond; therefore, these two protein fragments are covalently linked and form an irreversible complex. Additionally, the resilient interaction between these two binding partners can take place in a wide range of temperatures and pH.

In a preferred embodiment, the gene coding for SpyCatcher (²²D-N¹⁰³ from the CnaB2 domain) is fused in-frame to gC, gD, or gG ectodomains downstream of the signal peptide. Constructs for expression of SpyTag-antibodies are generated by synthesizing DNA sequences containing the coding sequences of SpyTag (AHIVMVDAYKPTK), a peptide linker (GSGGMHAAAAAGS) and an antibody against either CEACAM6 or EpCAM. These constructs are cloned into a pET22b vector, from which these proteins are fused with a c-terminal 6×His tag. After sequence confirmation, SpyTag-antibodies are expressed in the bacterial Rosetta (DE3) pLacI strain using IPTG induction, followed by purification by cobalt-bound HiTrap and size exclusion columns. Purified SpyTag-antibodies are added to the SpyCatcher-containing virus followed by passage through a gel filtration column to remove the free SpyTag-antibodies to obtain the oHSV decorated with antibodies against CEACAM6 or EpCAM.

In other embodiments, a bispecific antibody is used to target both a virus envelope protein and a tumor surface antigen. Bispecific antibodies combine the functionality and specificity of two antibodies in one molecule. The two antibodies are connected by a flexible linker and can simultaneously bind to their antigens. Therefore, bispecific antibodies place their targets into close proximity to facilitate subsequent biological events. In the present invention, bispecific antibodies are used to direct oHSV to cancer cells. In a preferred embodiment, the bispecific antibody is attached to the virus by binding to, e.g., glycoprotein D, which normally mediates tissue tropism. This serves to partially detarget the virus from its natural receptors, and renders the virus highly cancer-specific by retargeting its tropism via the tumor-specific antibody located on the other end of the bispecific antibody.

### C. Therapeutic Compositions

Therapeutic compositions are provided that may be used to prevent, treat, or ameliorate the effects of a disease, such as, for example, cancer. More particularly, therapeutic compositions are provided comprising at least one oncolytic virus as described herein, which has been decorated with a targeting moiety (e.g., a tumor specific antibody as described herein).

In certain embodiments, the compositions will further comprise a pharmaceutically acceptable carrier. The phrase “pharmaceutically acceptable carrier” is meant to encompass any carrier, diluent or excipient that does not interfere with the effectiveness of the biological activity of the oncolytic virus and that is not toxic to the subject to whom it is administered (see generally Remington: The Science and Practice of Pharmacy, Lippincott Williams & Wilkins; 21st ed. (May 1, 2005 and in The United States PharmacopE1A: The National Formulary (USP 40-NF 35 and Supplements).

In the case of an oncolytic virus as described herein, non-limiting examples of suitable pharmaceutical carriers include phosphate buffered saline solutions, water, emulsions (such as oil/water emulsions), various types of wetting agents, sterile solutions, and others. Additional pharmaceutically acceptable carriers include gels, bioadsorbable matrix materials, implantation elements containing the oncolytic virus, or any other suitable vehicle, delivery or dispensing means or material(s). Such carriers can be formulated by conventional methods and can be administered to the subject at an effective dose. Additional pharmaceutically acceptable excipients include, but are not limited to, water, saline, polyethyleneglycol, hyaluronic acid and ethanol. Pharmaceutically acceptable salts can also be included therein, e.g., mineral acid salts (such as hydrochlorides, hydrobromides, phosphates, sulfates, and the like) and the salts of organic acids (such as acetates, propionates, malonates, benzoates, and the like). Such pharmaceutically acceptable (pharmaceutical-grade) carriers, diluents and excipients that may be used to deliver the oHSV to a target cancer cell will preferably not induce an immune response in the individual (subject) receiving the composition (and will preferably be administered without undue toxicity).

The compositions provided herein can be provided at a variety of concentrations. For example, dosages of oncolytic virus can be provided which ranges from about 10⁶ to about 10⁹ pfu. Within further embodiments, the dosage can range from about 10⁶ to about 10⁸ pfu/ml, with up to 4 mls being injected into a patient with large lesions (e.g., >5 cm) and smaller amounts (e.g., up to 0.1 mls) in patients with small lesions (e.g., <0.5 cm) every 2-3 weeks, of treatment.

Within certain embodiments of the invention, lower dosages than standard may be utilized. Hence, within certain embodiments less than about 10⁶ pfu/ml (with up to 4 mls being injected into a patient every 2-3 weeks) can be administered to a patient.

The compositions may be stored at a temperature conducive to stable shelf-life, and includes room temperature (about 20° C.), 4° C., −20° C., −80° C., and in liquid N2. Because compositions intended for use in vivo generally don't have preservatives, storage will generally be at colder temperatures. Compositions may be stored dry (e.g., lyophilized) or in liquid form.

### D. Administration

In addition to the compositions described herein, various methods of using such compositions to treat or ameliorate cancer are provided, comprising the step of administering an effective dose or amount of a targeting moiety decorated OV vector as described herein to a subject.

The terms “effective dose” and “effective amount” refers to amounts of the oncolytic virus that is sufficient to effect treatment of a targeted cancer, e.g., amounts that are effective to reduce a targeted tumor size or load, or otherwise hinder the growth rate of targeted tumor cells. More particularly, such terms refer to amounts of oncolytic virus that is effective, at the necessary dosages and periods of treatment, to achieve a desired result. For example, in the context of treating a cancer, an effective amount of the compositions described herein is an amount that induces remission, reduces tumor burden, and/or prevents tumor spread or growth of the cancer. Effective amounts may vary according to factors such as the subject's disease state, age, gender, and weight, as well as the pharmaceutical formulation, the route of administration, and the like, but can nevertheless be routinely determined by one skilled in the art.

The therapeutic compositions are administered to a subject diagnosed with cancer or is suspected of having a cancer. Subjects may be human or non-human animals.

The compositions are used to treat cancer. The terms “treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

Representative forms of cancer include carcinomas, leukemia's, lymphomas, myelomas and sarcomas. Further examples include, but are not limited to cancer of the bile duct cancer, brain (e.g., glioblastoma), breast, cervix, colorectal, CNS (e.g., acoustic neuroma, astrocytoma, craniopharyogioma, ependymoma, glioblastoma, hemangioblastoma, medulloblastoma, menangioma, neuroblastoma, oligodendroglioma, pinealoma and retinoblastoma), endometrial lining, hematopoietic cells (e.g., leukemia's and lymphomas), kidney, larynx, lung, liver, oral cavity, ovaries, pancreas, prostate, skin (e.g., melanoma and squamous cell carcinoma), GI (e.g., esophagus, stomach, and colon) and thyroid. Cancers can comprise solid tumors (e.g., sarcomas such as fibrosarcoma, myxosarcoma, liposarcoma, chondrosarcoma and osteogenic sarcoma), be diffuse (e.g., leukemia's), or some combination of these (e.g., a metastatic cancer having both solid tumors and disseminated or diffuse cancer cells). Cancers can also be resistant to conventional treatment (e.g. conventional chemotherapy and/or radiation therapy).

Benign tumors and other conditions of unwanted cell proliferation may also be treated.

The OV (e.g., oHSV) as described herein may be given by a route that is e.g. oral, topical, parenteral, systemic, intravenous, intramuscular, intraocular, intrathecal, intratumor, subcutaneous, or transdermal. Within certain embodiments the oncolytic virus may be delivered by a cannula, by a catheter, or by direct injection. The site of administration may be intra-tumor or at a site distant from the tumor. The route of administration will often depend on the type of cancer being targeted.

The optimal or appropriate dosage regimen of the oncolytic virus is readily determinable within the skill of the art, by the attending physician based on patient data, patient observations, and various clinical factors, including for example a subject's size, body surface area, age, gender, and the particular oncolytic virus being administered, the time and route of administration, the type of cancer being treated, the general health of the patient, and other drug therapies to which the patient is being subjected. According to certain embodiments, treatment of a subject using the oncolytic virus described herein may be combined with additional types of therapy, such as chemotherapy using, e.g., a chemotherapeutic agent such as etoposide, ifosfamide, adriamycin, vincristine, doxycycline, and others.

OV (e.g., oHSV) may be formulated as medicaments and pharmaceutical compositions for clinical use and may be combined with a pharmaceutically acceptable carrier, diluent, excipient or adjuvant. The formulation will depend, at least in part, on the route of administration. Suitable formulations may comprise the virus and inhibitor in a sterile medium. The formulations can be fluid, gel, paste or solid forms. Formulations may be provided to a subject or medical professional

A therapeutically effective amount is preferably administered. This is an amount that is sufficient to show benefit to the subject. The actual amount administered and time-course of administration will depend at least in part on the nature of the cancer, the condition of the subject, site of delivery, and other factors.

Within yet other embodiments of the invention the oncolytic virus can be administered by a variety of methods, e.g., intratumorally, intravenously, or, after surgical resection of a tumor.

The following examples are offered by way of illustration, and not by way of limitation.

### EXAMPLES

### Example 1

### Generation of a High Affinity Anti-HSV-1 gG Single Domain Antibody

Anti-gG single domain antibodies, i.e., nanobodies, are generated by immunizing a llama, one of the few mammals known to produce heavy chain antibodies, with inactivated HSV-1 virus. The llama is immunized five times (days 1, 21, 35, 49, and 63) using 100 micrograms of viral protein per immunization. The immune response of the llama is monitored during the course of the immunizations using a standard ELISA protocol to ensure that specific heavy chain antibodies are generated. After the fifth immunization, peripheral blood mononuclear cells (PBMCs) are isolated from whole blood. RNA is extracted from the PBMCs using an RNA Blood Mini Kit (QIAGEN) and cDNA is then synthesized using a First-Strand cDNA Synthesis Kit (Thermo Fisher Scientific).

To clone gG-specific VHH domains of heavy chain antibodies generated during immunization, the variable domains of both VHH (600 bp) and VH (900 bp) are amplified from the PBMC cDNA by PCR, and the two pools of amplification products are separated by gel electrophoresis. The VHH amplification products are gel purified and a round of nested PCR is performed to re-amplify the entire repertoire of VHH gene fragments. The resulting PCR products are ligated into the phagemid vector, pMED1, which is then used to transform electrocompetent TG1 E. coli cells. The VHH repertoire is expressed in phage particles after rescue by co-infecting the cells with the M13K07 helper phage. VHH domains that specifically recognize the HSV-1 gG protein are enriched by performing two rounds of in vitro selection. To assess the enrichment of phage particles carrying antigen-specific VHHs, a serial dilution of the phages eluted from gC antigen-coated versus non-coated wells is used to transfect exponentially growing TG1 cells.

To enrich for target-specific nanobodies, two rounds of nanobody panning are performed using recombinant gG protein fragments. Three truncated forms of the gG ectodomain may be utilized, corresponding to amino acids 28-175 (SEQ ID NO:1), amino acids 57-175 (SEQ ID NO:2), and amino acids 84-175 (SEQ ID NO:3). Prior to use, purified protein fragments are tested to verify proper folding of the recombinant product. For nanobody panning, 0.5-1.0 mg of pure recombinant truncated gG ectodomain is used in each round. Individual colonies obtained after the second round of panning are tested against recombinant gG proteins in a phage ELISA. The unique VHH genes of the clones that scored positive in phage ELISA are sub-cloned into the bacterial expression vector pET22b, followed by expression and purification to obtain nanobodies with more than 95% demonstrable purity.

The binding affinity between recombinant gG and each nanobody is tested using the BLItz system (ForteBio), a standard biochemical device for measuring binding kinetics between antibodies and antigens. From these binding assays, the binding affinities of all nanobodies for recombinant gG are ranked against each other and the anti-gG nanobodies with the highest affinity are selected for the subsequent generation of bispecific antibodies.

### Example 2

### Assay for Determining the Ability of Antibody-Coated HSV-1 Virus to Infect Tumor Cells In Vitro

Wild-type HSV-1 is produced in Vero cells and virus-containing supernatant is collected three days post-infection. After passing the supernatant through a 0.45 micron filter, the filtered viral sample is mixed with different concentrations of commercially available anti-gG antibodies overnight at 4° C. Subsequently, experiments are performed to compare the infectivity of wild-type and anti-gG antibody-coated viruses in various tumor cell lines as described herein. After 3 days, infected tumor cell lysates are used for plaque assays on Vero cells to determine the level of virus production in the tested tumor cells.

### Example 3

### Generation of Bispecific Antibodies that Recognize Both HSV-1 and Tumor Antigens

After selection of several high affinity anti-HSV-1 gG nanobodies, wild-type HSV-1 viruses are decorated with each nanobody individually by mixing virus and purified nanobody protein in vitro. The infectivity of each nanobody-decorated viruses is tested to select the anti-gG nanobody that exhibits the smallest effect on virus infectivity.

Constructs for expression of bispecific antibodies are generated by linking the coding sequence of the selected anti-gG nanobody to that of an antibody directed against a tumor antigen (e.g., an anti-CEACAM6 or anti-EpCAM antibody). Linkage is mediated by a sequence encoding the peptide linker GGGGSGGGGSGGGGS (SEQ ID NO:4) or KRVAPELLGGPS (SEQ ID NO:5). The choice of peptide linker will depend upon the ability of the linker to maximize the structural flexibility of the bispecific antibody necessary for binding both target antigens, while maintaining a functional conformation. The bispecific antibody construct sequences are cloned into the pET22b vector for expression and purification.

### Example 4

### Determination of the Amount of Bispecific Antibody Bound to oHSV Per Virus Particle

oHSV is incubated in vitro with excess purified bispecific antibody, and the “decorated” (i.e., coated) virus is purified by gel filtration to remove any unbound antibody. qPCR is used to measure the number of viral particles in each tested lot, where a standard curve is created using a known amount of plasmid DNA carrying one of the viral genes.

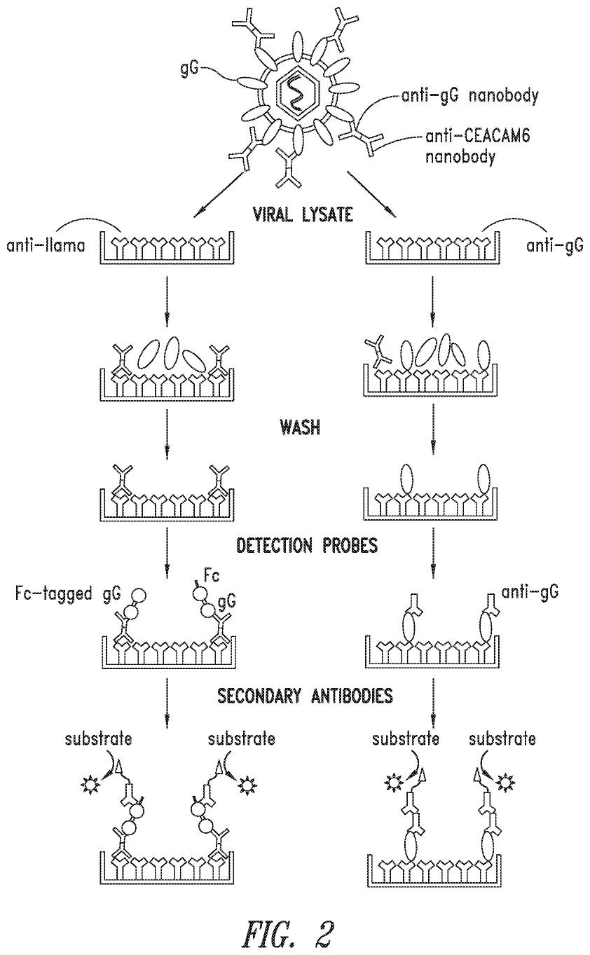

A sandwich ELISA assay (see FIG. 2) is used for measuring the amount of bispecific antibody and/or gG protein. On day 1, an ELISA plate is coated with a capture antibody, either against the bispecific antibody or gG, overnight at 4° C. On day 2, the lysates of ADOVs as shown in FIG. 2 are added to the plate and incubated at room temperature for 2 hours. Subsequently, a detection probe is used to bind to either the bound bispecific antibody or unbound gG for quantification (an illustration using anti-gG-anti-CEACAM6 bispecific antibody-coated oHSV is shown in FIG. 1). Using a standard with a known quantity (weight in grams), the amount of antibodies as well as gG molecules is calculated using the following formula: amount of the bispecific antibody or gG molecules=measured weight of the molecule (from ELISA assay)/molecular weight. Efficiency of bispecific antibody conjugation to gG is calculated by the following formula expressed as a percentage: average amount of the bispecific antibody bound/average amount of gG molecules×100%. The amount of conjugated bispecific antibody per virus particle is calculated with the following formula: the measured weight of the molecule/molecular weight*6.022×10²³)/number of virus particle used. Similar assays are performed at various time points (24, 48, 72 and 96 hours) after purification of ADOVs in both PBS and serum to measure stability of the bispecific antibody bound to ADOVs.

### Example 5

### In Vitro Testing of Bispecific Antibody Function

To test the abilities of the bispecific antibodies to bind both target antigens (e.g., HSV-1 gG and tumor-specific CEACAM6 or EpCAM), in vitro, binding assays are performed using the BLItz system. Briefly, the biosensor is coated with recombinant gG ectodomain protein. Then, the coated biosensor is contacted with the purified bispecific antibody, followed by contact with the purified CEACAM6 or EpCAM protein.

### Example 6

### Determination of the Amount of SpyTag-Antibody Bound to SpyCatcher-Decorated oHSV Per Virus Particle

oHSV decorated with SpyCatcher/SpyTag-antibody (anti-CEACAM6 or anti-EpCAM antibody) is generated and purified by incubating SpyCatcher-containing oHSV with purified SpyTag-fused antibodies, followed by removal of unconjugated SpyTag-fused antibody by gel filtration chromatography.

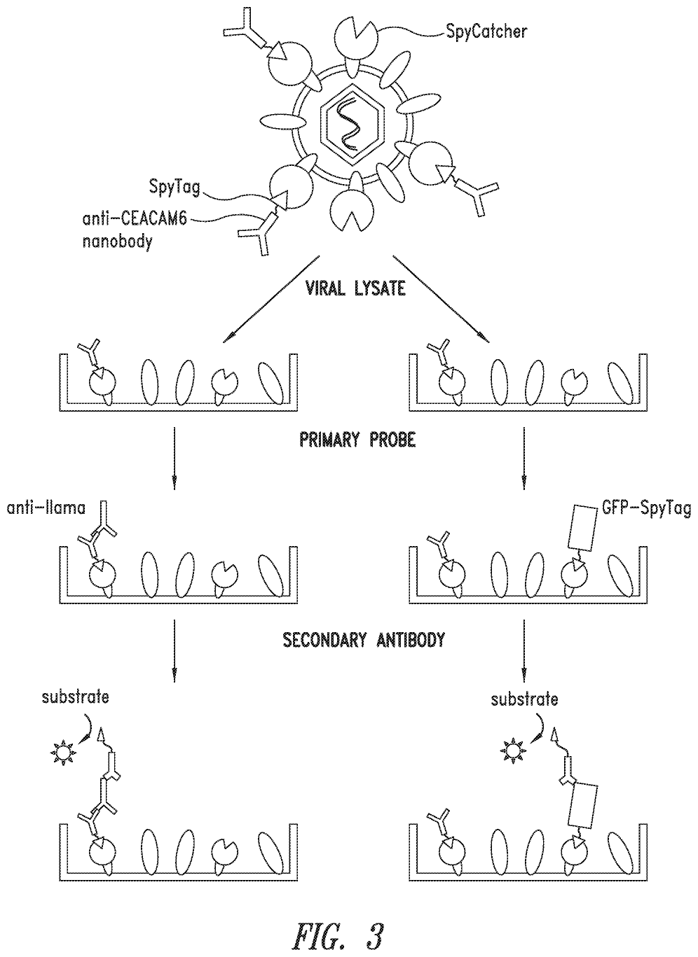

ELISA assays are performed to quantify the amount of SpyTag-antibody or SpyCatcher, as shown in FIG. 3. To measure bound SpyTag-antibody, a plate is coated with the lysates of SpyCatcher/SpyTag-decorated viruses. An antibody that recognizes the antibody conjugated to SpyTag is used as a primary antibody, followed by a secondary antibody for quantification. To measure unconjugated (free) SpyCatcher, a plate is coated with the same lysates of SpyCatcher/SpyTag-decorated viruses. GFP-tagged SpyTag that binds to unconjugated/free SpyCatcher is used as a “primary antibody”, followed by a secondary anti-GFP antibody for quantification (an illustration using SpyCatcher/SpyTag-anti-CEACAM6-coated oHSV is shown in FIG. 3). The GFP-tagged SpyCatcher is then cloned into an expression vector for expression and purification, as described herein. The efficiency of SpyTag-antibody conjugation to SpyCatcher and the amount of SpyTag-antibody bound to SpyCatcher decorated oHSV per virus particle is calculated using methods described herein.

### Example 7

### Verification of SpyCatcher-gC or SpyCatcher-gG Constructs

The SpyCatcher-gC or SpyCatcher-gG constructs are verified using a GFP-fused SpyTag. GFP-SpyTag is cloned into an expression vector in which the fusion protein is linked to a TEV (Tobacco Etch Virus) cleavage site, a 6×His tag and human Fc1 in sequence. After sequence confirmation, the recombinant GFP-SpyTag protein is expressed in a 2 liter suspension culture of Free-style 293 cells using PEI transfection. After 72 hours, the supernatant is collected and the fusion protein is purified by protein-G and HiTrap Cobalt columns in sequence, yielding 0.5-1.0 mg of pure GFP-SpyTag. Excess purified GFP-SpyTag is added to the SpyCatcher-gC or SpyCatcher-gG mutants and the mixture is run through a gel filtration column to remove the unbound GFP-SpyTag. To confirm that the virus has been successfully coated with GFP through the SpyCatcher/SpyTag system, flow cytometry is used measure the GFP⁺ virus after attaching the virus to permissive cells.

### Example 8

### Assay to Determine the Effects of Deletions in the Ectodomains of HSV-1 gG or gC on Oncolytic HSV-1 Infectivity

Mutant HSV-1 viruses with deletions in the ectodomain of gC or gG are constructed and used to infect a wide variety of tumor cell lines, including human lung cancer cells H460, breast cancer cells MDA-MB-231, colon cancer cells LS174, prostate cancer cells LNCAP, and bladder cancer cells UMUC3. For comparison, normal cells (e.g., human fibroblast cells purchased from ATCC) are also infected with the same mutants; the parental strain of HSV-1 is used as a control for infectivity. The cells are incubated with virus for 1 hour to allow for infection, followed by washing with PBS three times to remove any remaining extracellular virus. The total cell lysates are collected at 3 hours and 72 hours followed by qPCR to measure virus copy number. Viral copy numbers at 3 hours post infection shows the efficiency of viral attachment and entry into the cells, while the copy numbers at 72 hours post infection reflects the efficiency of viral replication and dissemination of progeny virus to adjacent cells. If no significant changes are observed in the infectivity or replication characteristics of the gG mutant, while only marginally reduced infectivity is observed in the gC mutant, it may be surmised that the latter may be caused by impaired binding of the virus to heparan sulfate on the host cell surface due to removal of the gC HS-binding domain.

### Example 9

### Tumor Specificity Testing of Antibody-Decorated oHSV In Vitro and In Vivo

Purified ADOVs and nondecorated oHSV-1 are incubated with a variety of tumor cells and nontumor cells. After 24 and 48 hours, infectivity and replication kinetics are tested using plaque assays. After 72 hours, the ability of the virus to kill tumor cells is tested by using an MTT cytotoxicity assay. Similar infectivity and cell killing is expected to be observed between ADOVs and their parental oncolytic viruses in vitro.

Human tumor-bearing nude or SCID mice are intravenously injected with various titers (10⁴ to 10′ pfu) of ADOVs or their parent virus via the tail vein. Animals are humanely euthanized at various time points (30 minutes, 1 hour, 4 hours, 12 hours, 24 hours and 72 hours) after injection and samples of tumor tissue and of tissues derived from all major organs are collected and analyzed by Q-PCR to quantify viral genome copy numbers so as to measure the bio-distribution of the virus. Higher virus copy numbers are expected to be found in tumor tissues relative to tissues derived from healthy organs.

For efficacy observations, some animals are allowed to survive longer after injection with ADOVs or the parental virus. Tumor sizes are measured every second day. Virus bio-distribution is also measured in animals after tumor regression following virus treatment. A smaller quantity of virus is expected to be necessary for the ADOVs, as compared to the parental virus, to achieve a similar level of tumor inhibition.

### Example 10

### Generation of a SpyTag-Anti-CEACAM6-Spycatcher Protein Conjugate

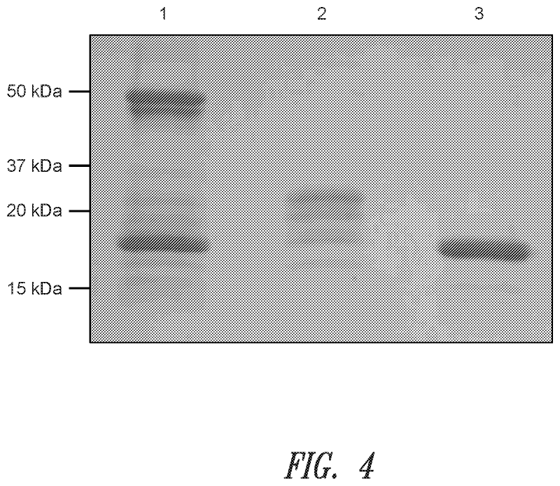

SpyTag-anti-CEACAM6 is a fusion protein comprising a single-domain antibody raised against the CEACAM6 tumor antigen fused to the SpyTag protein. To produce recombinant SpyTag-anti-CEACAM6 for conjugation, a SpyTag-anti-CEACAM6-Fc protein was expressed in FreeStyle 293 cells and purified using a protein G column, followed by TEV cleavage to remove the Fc tag. Recombinant SpyCatcher protein was expressed in E. coli BL21 DE3 pLysS cells and purified from cell lysates with cobalt-affinity columns. To conjugate the purified SpyCatcher and SpyTag-anti-CEACAM6 proteins, 5 μM Spycatcher was mixed with 10 μM SpyTag-anti-CEACAM6 and the mixture was incubated overnight at 4° C. Products of the conjugation reaction were analyzed by SDS/PAGE followed by Coomassie staining. As shown in FIG. 4, the SpyCatcher-SpyTag-anti-CEACAM6 conjugate (lane 1, top band) migrates as a higher molecular weight protein relative to the unconjugated SpyTag-anti-CEACAM6 (lane 2) and Spycatcher (lane 3) proteins.

### Example 11

### Quantification of the Binding of SpyTag-Anti-CEACAM6 to Virus Expressing SpyCatcher

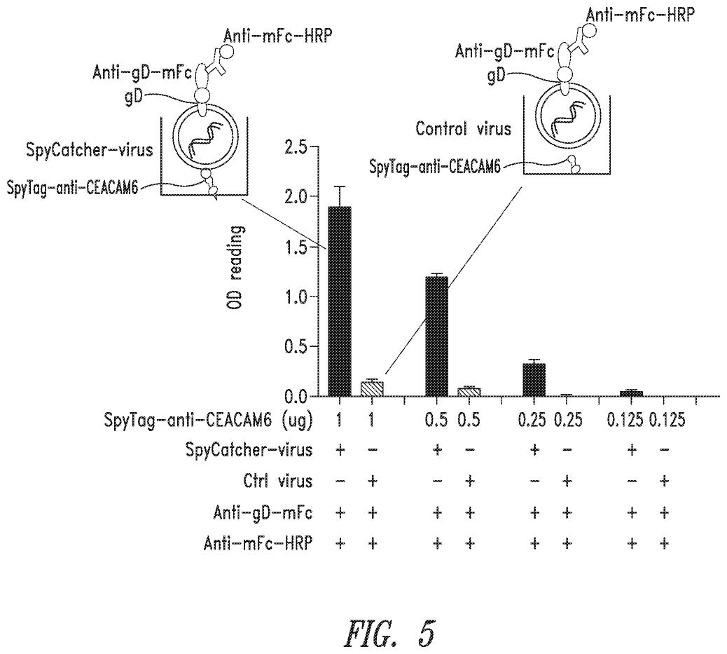

ELISA assays were performed to quantify the binding of SpyTag-anti-CEACAM6 to virus engineered to express the SpyCatcher protein. Briefly, different concentrations of recombinant SpyTag-anti-CEACAM6 were precoated onto a MaxiSorp ELISA plate and incubated overnight at 4° C. The following day, either a virus expressing SpyCatcher, or a control virus, was added to each well of the ELISA plate and the plate was incubated at room temperature for two hours. The plate was then washed and the binding of virus to the SpyTag-anti-CEACAM6 protein was detected by addition of mouse IgG Fc-conjugated anti-gD single domain antibody (anti-gD-mFc), HRP-conjugated anti-mouse IgG antibody (anti-mFc-HRP), and the substrate, 3,3′,5,5′-Tetramethylbenzidine (TMB), as illustrated in FIG. 5. Absorbance measurements were taken at 450 nm using a microplate reader (Molecular Devices). Results are shown in FIG. 5, which compares the amount of Spycatcher-expressing virus retained by the immobilized SpyTag-anti-CEACAM6 protein (represented by the solid bars) to that of the control virus that does not express SpyCatcher (represented by the hatched bars). These data indicate that virus engineered to express the SpyCatcher protein specifically binds the SpyTag-anti-CEACAM6 protein in a dose-dependent manner.

### Example 12

### In Vitro Viral Retargeting Mediated by the SpyTag-Anti-CEACAM6 Fusion Protein

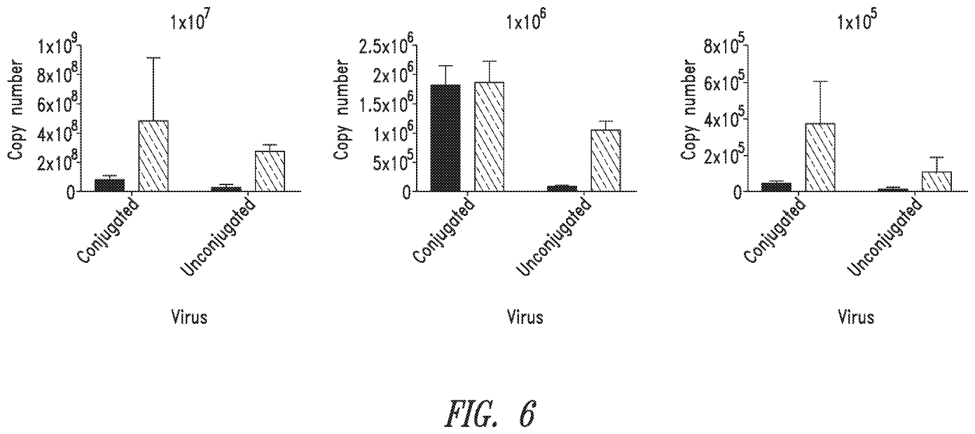

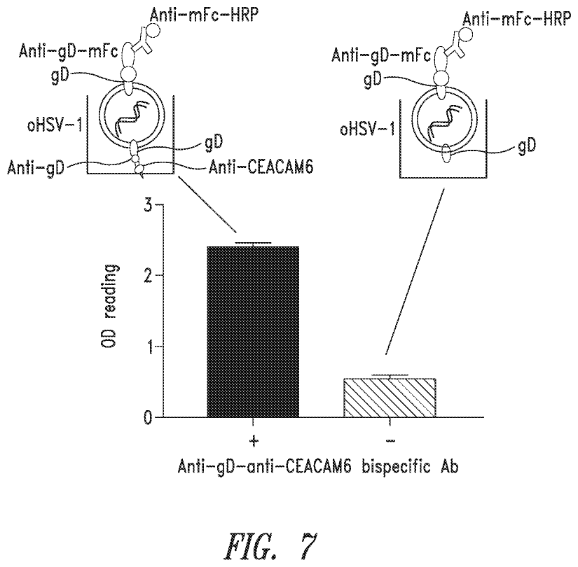

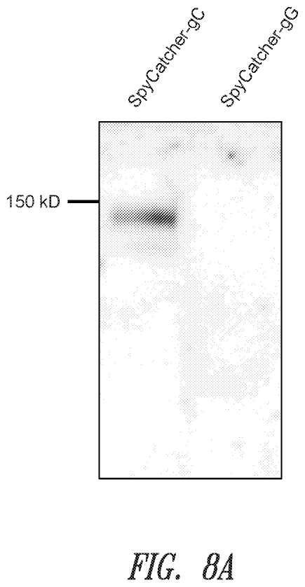

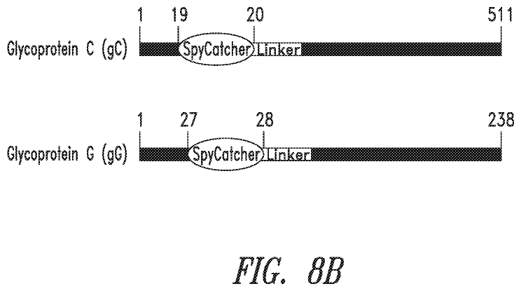

To generate virus coated with the SpyTag-anti-CEACAM6 fusion protein, virus engineered to express the Spycatcher protein was incubated with purified recombinant SpyTag-anti-CEACAM6 protein; unbound protein was removed by passing the mixture through a spin column. The ability of the coated virus to infect CT26-CEACAM6 cells was then assessed with serial dilutions of the virus. Briefly, viral dilutions were incubated with cells for one hour and unattached virus was then removed by washing the cells with PBS. Cells lysates were prepared and total DNA samples were extracted from each lysate after either 24 or 48 hours. Viral copy number was measured by qPCR performed on the purified viral DNA using primers designed to anneal to the viral ICP27 gene. As shown in FIG. 6, the SpyCatcher virus coated with the SpyTag-anti-CEACAM6 protein (corresponding to samples abbreviated, “anti-CEACAM6”) consistently generated higher viral copy numbers in CT26-CEACAM6 cells than the uncoated virus control (corresponding to samples abbreviated, “Spycatcher”). (The densely-hatched bars represent samples in which DNA was extracted after 24 hours, while the loosely-hatched bars represent samples in which DNA was extracted after 48 hours). These results indicate that the single domain antibody to CEACAM is capable of targeting virus to cells positive for the expression of the CEACAM6 tumor marker.

### Example 13

### Assessment of the Interaction Between o-HSV-1 and an Anti-gD-Anti-CEACAM6 Bispecific Antibody

To investigate the ability of o-HSV-1 to bind the anti-gD-anti-CEACAM6 bispecific antibody, ELISA assays were performed. Briefly, 0.25 μg of anti-gD-anti-CEACAM6 bispecific antibody was pre-coated onto MaxiSorp ELISA plate and then incubated at 4° C. overnight. The next day, oHSV-1 was added to each well and the plate was incubated at room temperature for 2 hours. The plate was then washed, and the interaction of oHSV-1 with the bispecific antibody was detected by mouse IgG Fc-conjugated to anti-gD single domain antibody (anti-gD-mFc), HRP-conjugated anti-mouse IgG antibody (anti-mFC-HRP), and the substrate, 3,3′,5,5′-Tetramethylbenzidine (TMB). Absorbance measurements were taken at 450 using a microplate reader (Molecular Devices). Results are shown in FIG. 7 and indicate that binding of the virus to the ELISA plate is highly dependent upon the presence of the immobilized anti-gD-anti-CEACAM6 bispecific antibody.

### Example 14

### Ability to Detect Fusion of SpyCatcher Protein to Viral Proteins

Expression constructs were made in which the coding regions of SpyCatcher were fused in-frame to that of the N-terminus of HSV-1 gC (starting at between amino acids 19 and 20) or the N-terminus of gG (starting at between amino acids 27 and 28). The C-terminus of SpyCatcher was joined to the remainder of the viral antigens by a peptide linker. Simplified illustrations of the resulting fusion proteins are presented in FIG. 8A. Vero cells were infected with viruses engineered to express either the SpyCatcher-gC or the SpyCatcher-gG fusion protein. After 48 hours, the cells were lysed in RIPA buffer on ice and the supernatant was collected after centrifugation at 15,000 rpm for 10 minutes. To detect the fusion proteins, Western blots were performed in which cell lysates were run on SDS-PAGE, followed by transferring to a nitrocellulose membrane. The membrane was probed with SpyTag-Neongreen-hFc, followed by a reaction with a horseradish peroxide-conjugated goat anti-human-Fc antibody. Results are shown in FIG. 8B. Surprisingly, SpyCatcher was only detectable in cells infected with the SpyCatcher-gC mutant virus. This suggests that the precise location of SpyCatcher insertion into the viral protein is critical to preserve SpyCatcher antigenicity.

The following are additional exemplary embodiments of the present disclosure:

1) An oncolytic virus which expresses one member of a covalent binding pair (CBP1).

2) The oncolytic virus according to embodiment 1 wherein said member of a covalent binding pair (CBP1) is encoded within a region of the virus not responsible for infection or replication. Within other embodiments of the invention, the CPB1 may play a role in viral infection or replication.

3) The oncolytic virus according to embodiment 2 wherein said CBP1 is encoded within any portion of the extracellular domain of the membrane-associated viral surface protein (e.g., an envelope protein) which is not responsible for infection of the oncolytic virus. Within other embodiments of the invention, the CBP1 is encoded within a portion of an extracellular domain which is responsible for infection of the oncolytic virus.

4) The oncolytic virus according to any one of embodiments 1 to 3 wherein said oncolytic virus is selected from the group consisting of oncolytic adenoviruses and oncolytic vaccinia viruses.

5) The oncolytic virus according to any one of embodiments 1 to 3 wherein said oncolytic virus is an oncolytic herpes virus.

6) The oncolytic viruses according to embodiment 5 wherein said oncolytic herpes virus envelope protein is selected from the group consisting of gC, gE, gG, gI, gJ, gM, gN, UL24, UL43, UL45, UL56, and US9. Within further embodiments the envelope protein is selected from a group that may play a role in viral infection or replication, e.g., gB, gD (US6), gK, and UL20. Within further embodiments of claims 1 to 6, representative examples of CPB include the SpyTag/SpyCatcher pair.

7) A bispecific antibody comprising two linked binding domains, wherein a first binding domain targets the envelope of an oncolytic virus, and a second binding domain targets a tumor specific antigen.

8) The bispecific antibody according to embodiment 7 wherein said envelope of an oncolytic virus is a herpes virus envelope.

9) The bispecific antibody according to embodiment 8 wherein said herpes virus envelope is selected from the group consisting of gC, gE, gG, gI, gJ, gM, gN, UL24, UL43, UL45, UL56, and US9. Within further embodiments the envelope protein is selected from a group which may play a role in viral infection or replication, e.g., gB, gD (US6), gK, and UL20.

10) A bispecific antibody comprising two linked binding domains, wherein a first binding domain is one member of a CBP, and the second domain targets a tumor antigen. Within various embodiments provided herein, representative examples of CPB include the SpyTag/SpyCatcher pair.

11) The bispecific antibody according to embodiment 10 wherein said first binding domain is SpyTag (AHIVMVDAYKPTK) (SEQ ID No. 6). Within other embodiments, the first binding domain is SpyCatcher.

12) The bispecific antibody according to any one of embodiments 7-11 wherein said tumor antigen is selected from the group consisting of CDK-1, COX-2, CRISP3, ESO-1, HER-2, MAGE-A1, MAGE-2, MAGE-A3, MAGE-86, MAPPK1, MICA, NY-ES-01, OLIG2, p53, ras, TRP-1, TRP-2, WT-1, XAGE2, and ZNF165

13) The bispecific antibody according to any one of embodiments 7 to 12 wherein said tumor antigen is CEACAM6 or EpCAM.

14) The bispecific antibody according to any one of embodiments 7 to 13 wherein said first binding domain is linked to said second binding domain with the peptide linker (GSGGMHAAAAAGS) (SEQ ID No. 7).

15) A composition comprising an oncolytic virus and a targeting moiety on the surface of said virus, wherein said oncolytic virus does not encode or express said one or more targeting moieties.

16) The composition of embodiment 15 wherein said oncolytic virus is selected from the group consisting of oncolytic adenoviruses, oncolytic herpes viruses and oncolytic vaccinia viruses.

17) The composition of embodiments 15 or 16 wherein said oncolytic virus expresses a member of a covalent binding pair (CBP). Within various embodiments, the CPB is the SpyTag/SpyCatcher pair.

18) The composition according to embodiment 17 wherein said member of a CPB is SpyCatcher. Within other embodiments, said member of a CPB is SpyTag.

19) The composition according to any one of embodiments 15 to 18 wherein said targeting moiety is an antibody.

20) The composition according to embodiment 19 wherein said antibody is a bispecific antibody.

21) The composition according to embodiment 19 wherein said bispecific antibody is a bispecific antibody according to any one of embodiments 7 to 14.

22) The composition according to any one of embodiments 15 to 21, further comprising an anti-glycoprotein D antibody. Within related embodiments of the invention the antibody blocks gD-mediated targeting of nectin-1, HVEM, and modified heparan sulfates which regulate HSV tropism.

23) A pharmaceutical composition comprising a composition according to any one of embodiments 1 to 14, along with a pharmaceutically acceptable excipient.

24) A pharmaceutical composition comprising a composition according to any one of embodiments 15 to 22, along with a pharmaceutically acceptable excipient.

25) A method for treating cancer, comprising administering to a patient a pharmaceutical composition according to embodiment 24. Within further embodiments the composition can be delivered by a variety of methods, e.g., intratumorally or intravenously.

26) The method according to embodiment 25, wherein said cancer is breast cancer, colon cancer or brain cancer.

27) The method of embodiment 25, further comprising the step of administering chemotherapy, radiotherapy and or immunotherapy prior to, during or subsequent to said step of administering a pharmaceutical composition according to any one of embodiments 15 to 21.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in the written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and applicants reserve the right to revise the application or claims to refer specifically to any individual member or any subgroup of members of the Markush group.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. An oncolytic virus which expresses one member of a covalent binding pair (CBP1).

2. The oncolytic virus according to claim 1 wherein said member of a covalent binding pair (CBP1) is encoded within a region of the virus not responsible for infection or replication.

3. The oncolytic virus according to claim 2 wherein said CBP1 is encoded within an envelope protein not responsible for infection of the oncolytic virus.

4. The oncolytic virus according to any one of claims 1 to 3 wherein said oncolytic virus is selected from the group consisting of oncolytic adenoviruses and oncolytic vaccinia viruses.

5. The oncolytic virus according to any one of claims 1 to 3 wherein said oncolytic virus is an oncolytic herpes virus.

6. The oncolytic viruses according to claim 5 wherein said oncolytic herpes virus envelope protein is selected from the group consisting of gB, gC, gD, gEgC, gD, gE, gG, gI, gJ, gK, gM, gN, UL20, UL24, UL43, UL45, UL56, and US9.

7. A bispecific antibody comprising two linked binding domains, wherein a first binding domain targets the envelope of an oncolytic virus, and a second binding domain targets a tumor specific antigen.

8. The bispecific antibody according to claim 7 wherein said envelope of an oncolytic virus is a herpes virus envelope.

9. The bispecific antibody according to claim 8 wherein said herpes virus envelope is selected from the group consisting of gB, gC, gD, gE, gG, gI, gJ, gK, gM, gN, UL20, UL24, UL43, UL45, UL56, and US9.

10. A bispecific antibody comprising two linked binding domains, wherein a first binding domain is one member of a covalent binding pair, and the second domain targets a tumor antigen.

11. The bispecific antibody according to claim 10 wherein said first binding domain is SpyTag (AHIVMVDAYKPTK) (SEQ ID No. 6).

12. The bispecific antibody according to any one of claims 7-11 wherein said tumor antigen is selected from the group consisting of CDK-1, COX-2, CRISP3, ESO-1, HER-2, MAGE-A1, MAGE-2, MAGE-A3, MAGE-86, MAPPK1, MICA, NY-ES-01, OLIG2, p53, ras, TRP-1, TRP-2, WT-1, XAGE2, and ZNF165.

13. The bispecific antibody according to any one of claims 7 to 12 wherein said tumor antigen is CEACAM6 or EpCAM.

14. The bispecific antibody according to any one of claims 7 to 13 wherein said first binding domain is linked to said second binding domain with the peptide linker (GSGGMHAAAAAGS) (SEQ ID No. 7).

15. A composition comprising an oncolytic virus and a targeting moiety on the surface of said virus, wherein said oncolytic virus does not encode or express said one or more targeting moieties.

16. The composition of claim 15 wherein said oncolytic virus is selected from the group consisting of oncolytic adenoviruses, oncolytic herpes viruses and oncolytic vaccinia viruses.

17. The composition of claims 15 or 16 wherein said oncolytic virus expresses a member of a covalent binding pair (CBP).

18. The composition according to claim 17 wherein said member of a CPB is SpyCatcher.

19. The composition according to any one of claims 15 to 18 wherein said targeting moiety is an antibody.

20. The composition according to claim 19 wherein said antibody is a bispecific antibody.

21. The composition according to claim 19 wherein said bispecific antibody is a bispecific antibody according to any one of claims 7 to 14.

22. A pharmaceutical composition comprising a composition according to any one of claims 1 to 14, along with a pharmaceutically acceptable excipient.

23. A pharmaceutical composition comprising a composition according to any one of claims 15 to 21, along with a pharmaceutically acceptable excipient.

24. A method for treating cancer, comprising administering to a patient a pharmaceutical composition according to claim 22 or 23.

25. The method according to claim 25, wherein said cancer is breast cancer, colon cancer or brain cancer.

26. The method of claim 25, further comprising the step of administering chemotherapy, radiotherapy and or immunotherapy prior to, during, or subsequent to said step of administering a pharmaceutical composition according to any one of claims 15 to 21.


SEQUENCE LISTING (7 sequences)

SEQ ID NO: 1 - protein, 148 aa, Herpes simplex virus type 1
NVSSTTQPQLQTTGRPSHEAPNMTQTGTTDSPTAISLTTPDHTPPMPSIGLEEEEEEEGAGDGEHLEGGDGTRDTLPQSPGPAFPLAEDVEKDKPNRPVVPSPDPNNSPARPETSRPKTPPTIIGPLATRPTTRLTSKGRPLVPTPQH

SEQ ID NO: 2 - protein, 119 aa, Herpes simplex virus type 1
DSPTAISLTTPDHTPPMPSIGLEEEEEEEGAGDGEHLEGGDGTRDTLPQSPGPAFPLAEDVEKDKPNRPVVPSPDPNNSPARPETSRPKTPPTIIGPLATRPTTRLTSKGRPLVPTPQH

SEQ ID NO: 3 - protein, 92 aa, Herpes simplex virus type 1
EEGAGDGEHLEGGDGTRDTLPQSPGPAFPLAEDVEKDKPNRPVVPSPDPNNSPARPETSRPKTPPTIIGPLATRPTTRLTSKGRPLVPTPQH

SEQ ID NO: 4 - protein, 15 aa, Artificial Sequence
  synthesized
GGGGSGGGGSGGGGS

SEQ ID NO: 5 - protein, 12 aa, Artificial Sequence
  synthesized
KRVAPELLGGPS

SEQ ID NO: 6 - protein, 13 aa, Streptococcus pyogenes
AHIVMVDAYKPTK

SEQ ID NO: 7 - protein, 13 aa, Artificial Sequence
  synthesized
GSGGMHAAAAAGS